(19) United States
(12) Patent Application Publication
(10) Pub. No.: US 2004/0167145 A1
(43) Pub. Date: Aug. 26, 2004

(54) Active ingredient combination for the pharmacological therapy of nicotine dependence

(76) Inventors: Klaus Opitz, Muenster (DE); Joachim Moormann, Werne (DE); Hermann Mucke, Wien (AT)

Correspondence Address: BIRCH STEWART KOLASCH & BIRCH, PO BOX 747, FALLS CHURCH, VA, 22040-0747, US

(21) Appl. No.: 10/482,961

(22) PCT Filed: Jul. 5, 2002
(86) PCT No.: PCT/EP02/07477

(30) Foreign Application Priority Data
Jul. 12, 2001 (DE) 10134038.9

(51) Int. Cl. A61K 31/55; A61K 31/485

(52) U.S. Cl. 514/282; 514/214.03

(57) ABSTRACT

The present invention relates to an active ingredient combination composed of at least one modulator of the cholinergic system with at least one substance blocking central opioid receptors for the pharmacological treatment of nicotine dependence.

The present invention relates to active ingredient combinations and to the use thereof for the pharmacological therapy of nicotine dependence, especially relating to cigarette consumption. In this connection, the active ingredient combination consists of at least one modulator of the cholinergic system with at least one substance which modulates the opioid receptor system. The present invention further relates to the use of the said active ingredient combination for producing medicaments which contribute to the therapy of nicotine consumption, in particular the consumption of cigarettes.

Intake of nicotine and related tobacco alkaloids through smoking with inhalation, and far less often by chewing or sniffing tobacco products, has central nervous stimulating effects which derive in particular from stimulation of the cholinergic and dopaminergic conduction systems. According to the current state of knowledge, this is attributable to a functional activation and increased expression of the presynaptic “nicotinic” acetylcholine receptors (nAChR), on which not only the natural agonist acetylcholine but also nicotine acts in the same way, and can bring about increased release of the relevant neurotransmitters (acetylcholine and dopamine). Inhibition of monoamine oxidase (MAO) enzymes by tobacco products acts in the same way, namely in the direction of increasing the intrasynaptic dopamine level; heavy smokers exhibit a reduction of 20-40% in the activity of MAO-A and MAO-B (see, for example, Berlin and Anthenelli, Int. J. Neuropsychopharmacol. 2001; p 4(1):33-42). Interaction of these cholinergic and dopaminergic factors brings about a considerable part of the cognitive and mood-lightening, rewarding effects desired by the smoker (concerning this, see Volodymyr et al., Nature 1997; Vol. 390, 401-404), but other conduction systems which use noradrenaline, serotonin, gamma-aminobutyric acid (GABA) and, in particular, also peptides which have an opioid-like effect as neurotransmitters are also modulated directly or indirectly and brought into a new neurobiological equilibrium which is different from that of the nonsmoker.

Heavy smoking which continues for many years is known to lead to a plethora of serious functional disorders, which are associated with considerable mortality, of the lungs and the cardiovascular system, and to an increased incidence of certain carcinomas. In nations with developed health systems, smoking is currently the commonest cause of statistically premature deaths. The calculations for Germany show 110,000 deaths caused directly by nicotine, and 80 million marks annually in associated costs.

However, because of the nicotine-induced neurophysiological changes mentioned above, the attempt to restrict or stop smoking gives rise to considerable withdrawal symptoms. In fact, the success of treatment of nicotine dependence, with permanent abstinence rates of between 10 and 35% overall, still lags behind the results for alcohol dependence. Medical persuasion on its own is successful in only about 5% of cases. Pharmacological replacement by nicotine plasters has permanent success rates of 10-15%, which can be increased by additional behavioural therapy in the best case to 30-35%. However, it must be remembered in this connection that although transdermal administration of nicotine eliminates the intake of carcinogens in the smoke, there is no effect at all on the cardiovascular risks caused directly by nicotine.

A nicotine-free oral replacement is available in the form of the active ingredient bupropion (Zyban®, GlaxoSmithKline) which acts at the noradrenergic and dopaminergic level and in clinical studies has achieved a 1-year abstinence rate of 28% (compared with 8% for placebo) and is not significantly more effective than transdermal nicotine in other parameters of achieving abstinence from smoking either.

There is thus an unaltered need for a therapy which is not based on direct nicotine replacement and which assists in reducing tobacco consumption in a harmless way with as few side effects as possible. For this reason there has been no lack of attempts over the years to introduce pharmacological improvements into the achieving of abstinence from smoking, particular attention being directed not only at cholinergic modulators but also at opioid antagonists because the mechanisms bringing about the desire for the substance (craving) are regarded as being located in the opioid system.

As an alternative to assisting with the withdrawal of nicotine by means of cholinergic modulators, the publications DE 43 01 782 (equivalent to EP 0 680 326 and U.S. Pat. No. 5,643,905) for example propose the use of galanthamine, which is said to suppress the desire for nicotine. This applies in the same way to deoxypeganine, which is claimed for this purpose in DE 199 06 979 (equivalent to WO 00 48 445) and, because of its simultaneous inhibition of monoamine oxidases, has a particularly high therapeutic potential.

In addition, U.S. Pat. No. 5,932,238 describes a transdermal therapeutic system suitable for galanthamine.

Galanthamine is also used for the treatment of poliomyelitis, of Alzheimer's disease and of various disorders of the nervous system, and for the treatment of closed-angle glaucoma.

Galanthamine or galantamine (4a,5,9,10,11,12-Hexahydro-3-methoxy-11-methyl-6-H-benzofuro(3a,3,2-ef)-(2)-benzazepin-6-ol) is a tetracyclic alkaloid which occurs in certain plants, especially in amaryllidaceae. It can be isolated from these plants by known processes (for example as disclosed in DE 195 09 663 A1 or DE-PS 11 93 061) or by a synthetic route (for example Kametani et al., J. Chem. Soc. C. 6, 1043-1047 (1971) or Shimizu et al., Heterocycles 8, 277-282 (1977)).

On the basis of its pharmacological properties, galanthamine is included in the group of reversibly acting cholinesterase inhibitors. At the same time, galanthamine also stimulates the release of the neurotransmitter acetylcholine through direct stimulation of the presynaptic nicotinic acetylcholine receptors. An analogous process also takes place at dopaminergic presynaptic nerve endings, where it promotes the release of dopamine. These properties of galanthamine are said according to current theories to reduce the craving independently of cognitive control. This forms the theoretical basis for the publications DE 40 10 079 and U.S. Pat. No. 5,932,238, which relate to the therapy of alcohol dependence and the symptoms of alcohol withdrawal, and is also mentioned in the patent DE 101 29 265.1, which describes combinations of galanthamine with inhibitors of neuroexcitatory processes in alcohol abuse.

The combined direct cholinergic and indirect dopaminergic effect described for galanthamine can also be achieved with substances which simultaneously inhibit acetylcholinesterase and monoamine oxidase. This is the case for example with deoxypeganine which is also referred to as deoxyvasicine, especially in the older literature. It was additionally proposed to use deoxypeganine likewise for the treatment of nicotine dependence through reducing the desire for nicotine or for replacement therapy of drug addicts and for the treatment of withdrawal symptoms during withdrawal therapy (WO 00 48 582), and for the pharmacological therapy of alcohol abuse and Alzheimer's dementia. In addition, deoxypeganine can, as cholinesterase inhibitor, be employed as antidote or prophylactic in cases of poisoning by organic phosphates, in which case it antagonizes the cerebral effect of cholinergic poisons.

Deoxypeganine (1,2,3,9-tetrahydropyrrolo[2,1-b]-quinazoline) is an alkaloid of molecular formula C₁₁H₁₂N₂ which is present in plants of the zygophyllaceae family. Deoxypeganine is preferably obtained by isolation from Syrian rue (Peganum harmala) or by synthesis.

Despite their duplicated mechanisms of action, galanthamine and deoxypeganine have only restricted suitability for effective suppression of the desire to smoke. The reason for this is likely to be that the desire for tobacco consumption is, according to the current state of knowledge, crucially caused in part by the endogenous opioid system activated by regular smoking.

Opioid receptor antagonists, some of which have been used clinically for a considerable time in the withdrawal therapy of alcohol and opiate abuse, have therefore likewise been proposed to assist in achieving abstinence from smoking, for example the closely related active ingredients naltrexone, naloxone and nalbuphine in oral formulations or in the form of transdermal therapeutic systems (U.S. Pat. No. 6,004,970, U.S. Pat. No. 4,573,995), and similarly nalmefene (U.S. Pat. No. 5,852,032). The same applies to the 5,9-dimethylbenzomorphanes cyclazocine (U.S. Pat. No. 5,965,567, Maisonneuve and Glick, NeuroReport 1999; 10: 693-696) and pentazocine. These show a differentiated spectrum of effects (antagonistic on mu opioid receptors and agonistic on kappa opioid receptors, likewise modulation of sigma receptors).

Since, of these substances, the most comprehensive data are available on the use of naltrexone in humans, it has also been investigated the most intensively for controlling the desire to smoke. The results obtained thereby are thoroughly contradictory. Although case reports and smaller studies showed that naltrexone is able in certain circumstances to reduce the enjoyment of smoking and the number of cigarettes smoked a day (Psychopharmacology 1998; 140(2): 185-190 and J. Clin. Psychiatry 1998; 59(1): 30-31 and Pharmacol. Biochem. Behav. 2000; 66(3): 563-572), the results of three randomized clinical studies on a total of 180 smokers were negative (Psychopharmacology 1995; 120(4): 418-425, Addiction 1999; 94(8): 1227-1237 and J. Addict. Dis. 1999; 18(1): 31-40). Likewise, data indicating that naltrexone in combination with transdermally administered nicotine depresses the stimulus to smoke (Psychopharmacology 1999; 142(2): 139-143), contrast with other results showing that naltrexone in fact abolishes the tobacco-avoiding effect of previously applied nicotine plasters (Psychopharmacology 1999; 143(4): 339-346), which is consistent with data from earlier animal experiments. Findings from electrophysiological in vitro experiments suggesting that naltrexone influences certain subtypes of nicotinic receptors in the brain in different ways in relation to activity and expression (Neuropharmacology 200; 39(13), 2740-2755) may possibly serve as a partial explanation of the latter phenomenon, as well as individual differences in the uptake of naltrexone given orally and the concentrations thereof reached in the brain.

Thus, overall, the reduction in tobacco consumption is not achieved in a satisfactory manner either by administration only of modulators of nicotinic receptors or by administration only of opioid receptor antagonists employed in alcohol withdrawal. The aim of the present invention was therefore to provide active ingredient combinations for producing medicaments by which the desire to smoke is depressed better than by the methods described above without, however, causing side effects which in turn increase the desire to smoke caused by increased stress.

It has surprisingly been found that the object on which the present invention is based can be solved particularly well by the combination of certain substances acting as modulators of the cholinergic system with substances which primarily act as opioid receptor antagonists.

The modulators of the cholinergic system which are used according to the invention, besides their inhibitory effect on cholinesterases, also act on dopaminergic nerve endings. This is possible for example with substances which, as cholinesterase inhibitors, also directly stimulate nicotinic acetylcholine receptors at the presynaptic nerve endings of cholinergic and dopaminergic nerve endings, or with substances which simultaneously inhibit acetylcholin-esterase and monoamine oxidase.

The modulators of the cholinergic system having the properties mentioned above which are preferably used are galanthamine or deoxypeganine or pharmacologically acceptable derivatives thereof. It is self-evident to the skilled person that galanthamine or deoxypeganine are used in the form of their free bases or in the form of their known salts or derivatives. Thus, for example, in place of the salts or addition compounds of galanthamine it is also possible to use all galanthamine derivatives mentioned or claimed in the scientific literature and in patents as long as they are either inhibitors of cholinesterase enzymes or modulators of nicotinic acetylcholine receptors, or combine both pharmacological activities. These include, in particular:

The compounds mentioned in the patents of the families WO-9612692/EP-0787115/U.S. Pat. No. 6,043,359 and WO-9740049/EP-0897387 and WO-032199 (Waldheim Pharmazeutika GmbH. and Sanochemia Pharmazeutika AG), including, in particular:

(−)-N-Demethylgalanthamine;

(−)-(N-Demethyl)-N-allylgalanthamine;

(−)-(6-Demethoxy)-6-hydroxygalanthamine (SPH-1088);

(±) N-Demethylgalanthamine N-tert-butyl carboxamide (SPH-1221);

(−) N-Demethylgalanthamine N-tert-butyl carboxamide

The compounds mentioned in the patents of the families EP-0648771 and EP-0653427 (Hoechst Roussel Pharmaceuticals Inc.) and Drugs Fut. 21(6), 621-635 (1996) and J. Pharmacol. Exp. Ther. 277(2), 728-738 (1996), including, in particular:

(−)-6-O-Demethylgalanthamine;

(−)-(6-O-Acetyl)-6-O-demethylgalanthamine (P11012);

(−)-(6-O-Demethyl)-6-O-[(adamantan-1-yl)carbonyl]galanthamine (P11149);

(−)-(6-O-Demethyl)-6-O-(triethylsilyl)galanthamine;

(−)-(6-O-Demethyl)-6-O-(triisopropylsilyl)galanthamine;

(−)-(6-O-Demethyl)-6-O-(trimethylsilyl)galanthamine;

The compounds mentioned in the patents of the families WO-9703987/EP-0839149/U.S. Pat. No. 5,958,903 (Societe de Conseils de Recherches et D'Applications Scientifiques, S.C.R.A.S) including, in particular:

(6-O-Demethyl)-6-O-(8′-phthalimidooctyl)galanthaminium bromohydrate;

(6-O-Demethyl)-6-O-(4′-phthalimidobutyl)galanthaminium bromohydrate;

(6-O-Demethyl)-6-O-(10′-phthalimidodecyl)galanthaminium bromohydrate;

(6-O-Demethyl)-6-O-(12′-phthalimidododecyl)galanthaminium bromohydrate;

10-N-Demethyl-10-N-(10′-phthalimidobutyl)galanthaminium trifluoroacetate;

10-N-Demethyl-10-N-(10′-phthalimidohexyl)galanthaminium trifluoroacetate;

10-N-Demethyl-10-N-(10′-phthalimidooctyl)galanthaminium bromohydrate;

10-N-Demethyl-10-N-(10′-phthalimidododecyl)galanthaminium bromohydrate;

10-N-Demethyl-10-N-(12′-phthalimidododecyl)galanthaminium bromohydrate;

10-N-Demethyl-10-N-(6′-pyrrolohexyl)galanthaminium bromohydrate

The (−)N,N′-demethyl-N,N′-bisgalantamine derivatives, which are described inter alia in the publication Bioorg. Med. Chem. 6(10), 1835-1850 (1998), of the following structural formula, where the bridging group (“alkyl spacer”) between the nitrogen atoms of the two galanthamine molecules may be 3-10 CH₂ groups long and, independently thereof, one of the two galanthamine molecules may carry a positive charge on the nitrogen atom (galanthaminium cation):

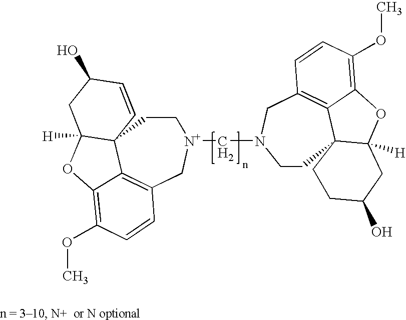

The (−)N-demethyl-N-(3-piperidinopropyl)galanthamine (SPH-1286), which is described inter alia in the publication J. Cerebral Blood Flow Metab. 19(Suppl. 1), S19 (1999) and in Proteins 42, 182-191 (2001), and its analogues with alkyl spacers up to 10 CH₂ groups long:

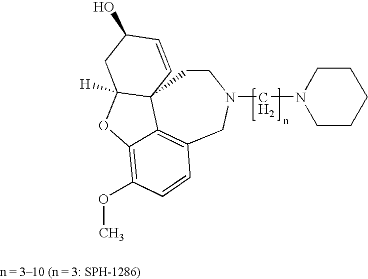

In place of deoxypeganine, its derivatives described in the literature are also to be understood in a similar way as long as they are simultaneously inhibitors of acetylcholinesterase and of monoamine oxidases. These include the 7-bromodeoxypeganine described in Synthetic Communs. 25(4), 569-572 (1995), as well as the 7-halo-6-hydroxy-5-methoxydeoxypeganines which are described in Drug Des. Disc. 14, 1-14 (1996) and have the general formula

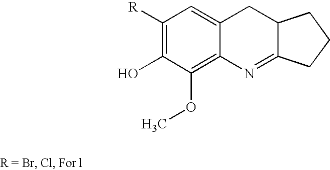

7-Bromo-6-hydroxy-5-methoxydeoxypeganine

7-Chloro-6-hydroxy-5-methoxydeoxypeganine

7-Fluoro-6-hydroxy-5-methoxydeoxypeganine

7-Iodo-6-hydroxy-5-methoxydeoxypeganine

The deoxypeganine derivatives described in Ind. J. Chem. 24B, 789-790 (1985) can also furthermore be used, namely 1,2,3,9-tetrahydro-6,7-methylenedioxypyrrolo[2,1-b]-quinazoline and 2,3-dihydro-6,7-dimethoxypyrrolo[2,1-b]-quinazoline-9(1H)-one.

The administered single dose of galanthamine or one of its pharmacologically acceptable salts or derivatives is preferably in the range from 1 to 50 mg, whereas the administered single dose of deoxypeganine or one of its pharmacologically acceptable salts or derivatives is preferably in the range from 10 to 500 mg.

According to the invention, galanthamine or deoxypeganine or one of their pharmacologically acceptable salts or derivatives are combined with at least one substance displaying antagonistic effects on opioid receptors.

The object is achieved particularly advantageously by a combination with representatives of particular opioid receptor antagonists and pharmacologically acceptable compounds. These include in particular

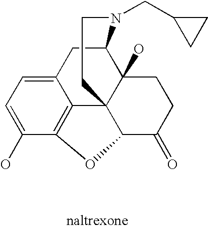

4,5-Epoxy-17-(cyclopropylmethyl)-3,14-dihydroxymorphinan-6-one

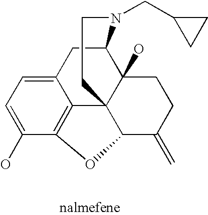

4,5-Epoxy-5-alpha-17-(cyclopropylmethyl)-6-methylene-morphinan-3,14-diol

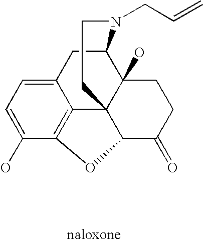

4,5-Epoxy-3,14-dihydroxy-17-(2-propenyl)morphinan-6-one

and nalorphine and nalbuphine.

It is evident that these substances can be used in the form of all their pharmacologically acceptable salts and addition compounds. Thus, naltrexone can also be employed as hydrobromide etc. in place of the hydrochloride which is mostly used. It is likewise evident that it is also possible in place of the substances mentioned above to employ the derivatives thereof having comparable pharmacological activity, especially all those claimed in WO 0 112 196 (Southern Research Institute), which include in particular the following naltrexone derivative:

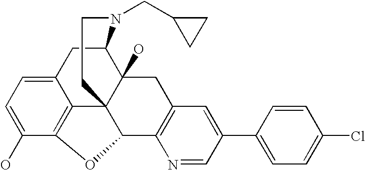

5′-(4-Chlorophenyl)-17-(cyclopropylmethyl)-6,7-didehydro-3,14-dihydroxy-4,5α-epoxypyrido[2′,3′:6,7]morphinan

The administered single dose of naltrexone or one of its pharmacologically acceptable salts or derivatives is preferably in the range from 1 to 200 mg.

It is likewise possible to employ the opioid receptor modulator cyclazocine in its two stereoisomeric forms ((+) and (−)) and as racemic mixture, likewise pentazocine. The administered single dose of cyclazocine or pentazocine or one of its pharmacologically acceptable salts or derivatives is preferably in the range from 5 to 100 mg.

The pharmaceutical forms which can be used according to the present invention for administering a combination of modulators of the cholinergic system with a substance acting as opioid receptor antagonist or opioid receptor modulator may comprise one or more of the following additives:

antioxidants, synergists, stabilizers;

preservatives;

taste-masking agents;

colours;

solvents, solubilizers;

surfactants (emulsifiers, solubilizers, wetting agents, antifoams);

agents affecting the viscosity and consistency, gel formers;

absorption promoters;

adsorbents, humectants, glidants;

agents affecting disintegration and dissolution, fillers (extenders), peptizers;

release-delaying agents.

This list is not definitive; the suitable physiologically acceptable substances are known to the skilled person.

A combination of modulators of the cholinergic system with opioid receptor antagonists or modulators can be administered orally or parenterally. It is possible to use medicaments in known dosage forms such as tablets, coated tablets or pastilles for oral administration. Also suitable are liquid or semiliquid dosage forms, in which case the active ingredient is in the form of a solution or suspension. Solvents or suspending agents which can be used are water, aqueous media or pharmacologically acceptable oils (vegetable or mineral oils).

The medicaments containing a combination of modulators of the cholinergic system with an opioid receptor antagonist or modulator are preferably formulated as depot medicaments which are able to deliver this active ingredient to the body in a controlled manner over a prolonged period.

It is also possible according to the invention for a combination of modulators of the cholinergic system with an opioid receptor antagonist or modulator to be administered by the parenteral route. For this purpose it is particularly advantageous to use transdermal or transmucosal dosage forms for the administration according to the invention of a combination of modulators of the cholinergic system with an opioid receptor antagonist or modulator, in particular adhesive transdermal therapeutic systems (active-ingredient plasters). These make it possible to deliver the active ingredient in a controlled manner over a prolonged period via the skin to the patient to be treated.

A further advantage is that misuse is less easily possible with parenteral administration forms than with oral dosage forms. The predetermined active ingredient-release area and the predetermined release rate mean that overdosage by the patient can be substantially ruled out. In addition, transdermal dosage forms are very advantageous because of other properties, e.g. avoidance of the first-pass effect or a better, more uniform control of the blood level.

Such transdermal systems containing a combination of modulators of the cholinergic system with an opioid receptor antagonist or modulator normally have an active ingredient-containing, contact adhesive polymer matrix which is covered on the side remote from the skin by an active ingredient-impermeable backing, and whose adhesive, active ingredient-delivering surface is covered before application by a detachable protective layer. The manufacture of such systems and the basic materials and excipients which can be used therefor are known in principle to the skilled person; for example, the assembly of such transdermal therapeutic systems is described in German patents DE 33 15 272 and DE 38 43 239 or in U.S. Pat. Nos. 4,769,028, 5,089,267, 3,742,951, 3,797,494, 3,996,934 and 4,031,894.

The combination, according to the invention, of a modulator of the cholinergic system with an opioid receptor antagonist or modulator can be used in achieving abstinence from nicotine in order to reduce the consumption of tobacco products, especially that of cigarettes but also of chewing tobacco.

The object of the invention is achieved in an illustrative manner as follows, it not being intended to restrict the scope of the invention by this illustrative list.

### EXAMPLE 1

Medicament to be administered orally or transdermally and containing 1 mg to 50 mg of galanthamine in the form of one of its pharmacologically acceptable salts, preferably in the form of its hydrobromide, or addition compounds and 10 mg to 100 mg of naltrexone, preferably in the form of the hydrochloride, per single dose.

### EXAMPLE 2

Medicament to be administered orally or transdermally and containing 10 mg to 500 mg of deoxypeganine in the form of one of its pharmacologically acceptable salts, preferably in the form of its hydrochloride, or addition compounds and 10 mg to 100 mg of naltrexone, preferably in the form of the hydrochloride, per single dose.

1. Active ingredient combination composed of at least one inhibitor of cholinesterase, which inhibitor also acts on dopaminergic nerve endings, with at least one substance modulating the opioid receptor system for the pharmacological therapy of nicotine dependence.

2. Active ingredient combination according to claim 1, characterized in that the inhibitor of cholinesterase is selected from the group comprising galanthamine and deoxypeganine in the form of their free base, their salts and addition compounds and the pharmacologically acceptable derivatives thereof.

3. Active ingredient combination according to claim 1 or 2, characterized in that the substance modulating the opioid receptor system or at least one of the substances modulating the opioid receptor system is selected from the group comprising naltrexone, nalmefene, naloxone, nalorphine, nalbuphine and the pharmacologically acceptable salts, derivatives and addition compounds thereof.

4. Active ingredient combination according to claim 3, characterized in that the substance modulating the opioid receptor system or at least one of the substances modulating the opioid receptor system is preferably selected from the group comprising naltrexone hydrochloride, naltrexone hydrobromide and 5′-(4-chlorophenyl)-17-(cyclopropylmethyl)-6,7-didehydro-3,14-dihydroxy-4,5α-epoxypyrido[2′,3′:6,7]morphinan.

5. Active ingredient combination according to claim 1 or 2, characterized in that the substance modulating the opioid receptor system or at least one of the substances modulating the opioid receptor system is selected from the group comprising cyclazocine and pentazocine in each of their two stereoisomeric forms and as mixture, and the pharmacologically acceptable salts and derivatives thereof.

6. Active ingredient combination according to any of the preceding claims, characterized in that it is in the form of a pharmaceutical form where the single dose of galanthamine, its pharmacologically acceptable salts, addition compounds or derivatives to be administered is preferably in a range from 1-50 mg, or the single dose of deoxypeganine or its pharmacologically acceptable salts, addition compounds or derivatives to be administered is preferably in a range from 10-500 mg.

7. Active ingredient combination according to any of the preceding claims, characterized in that it is in the form of a pharmaceutical form where the single dose of naltrexone, its pharmacologically acceptable salts, addition compounds or derivatives to be administered is preferably in a range from 1 to 200 mg, or the single dose of cyclazocine or pentazocine, their pharmacologically acceptable salts or derivatives to be administered is preferably in a range from 5 to 100 mg.

8. Active ingredient combination according to any of the preceding claims, characterized in that it is in the form of a pharmaceutical form which has a depot effect.

9. Active ingredient combination according to any of the preceding claims, characterized in that it is in the form of a medicament to be administered orally.

10. Active ingredient combination according to any of the preceding claims, characterized in that it is in the form of a medicament to be administered parenterally.

11. Active ingredient combination according to claim 10, characterized in that it is in the form of a medicament to be administered transdermally.

12. Use of an active ingredient combination according to any of claims 1 to 5 for pharmacological therapy of nicotine dependence.

13. Use of an active ingredient combination according to any of claims 1 to 5 for producing a pharmaceutical form for pharmacological therapy of nicotine dependence.

14. Use according to claim 12 or 13, characterized in that the pharmaceutical form is produced in the form of an oral dosage form.

15. Use according to claim 12 or 13, characterized in that the pharmaceutical form is produced in the form of a parenteral dosage form.

16. Use according to claim 15, characterized in that the pharmaceutical form is produced in the form of a transdermal dosage form.

17. Use according to any of claims 12 to 16, characterized in that the pharmaceutical form comprises a single dose for administration of galanthamine, its pharmacologically acceptable salts, addition compounds or derivatives preferably in a range from 1 to 50 mg, or of deoxypeganine, its pharmacologically acceptable salts, addition compounds or derivatives preferably in a range from 10 to 500 mg.

18. Use according to any of claims 12 to 17, characterized in that the pharmaceutical form comprises a single dose for administration of naltrexone, its pharmacologically acceptable salts, addition compounds or derivatives preferably in a range from 1 to 200 mg, or of cyclazocine or pentazocine or their pharmacologically acceptable salts or derivatives preferably in a range from 5 to 100 mg.

19. Method for the pharmacological therapy of nicotine dependence, characterized in that an active ingredient combination according to one or more of claims 1 to 5 is administered.
